(19) United States
(12) Patent Application Publication
(10) Pub. No.: US 2025/0134783 A1
(43) Pub. Date: May 1, 2025

(54) ORAL CARE COMPOSITION COMPRISING POROUS SILICA PARTICLES

(71) Applicant: Sigrid Therapeutics AB, Stockholm (SE)

(72) Inventors: Jeanha Baek, Stockholm (SE); Tore Bengtsson, Vaxholm (SE); Anna Ioannidou, Stockholm (SE); Eric Johnston, Bromma (SE); Ghislaine Monique Nicole Robert-Nicoud, Stockholm (SE)

(21) Appl. No.: 18/835,391

(22) PCT Filed: Feb. 3, 2023
(86) PCT No.: PCT/EP2023/052641

§ 371 Date: Aug. 2, 2024

(30) Foreign Application Priority Data
Feb. 4, 2022 (GB) 2201484.9

(51) Int. Cl. A61K 8/02; A61K 8/25; A61Q 11/00

(52) U.S. Cl. CPC A61K 8/0279; A61K 8/25; A61Q 11/00; A61K 2800/28; A61K 2800/413; A61K 2800/651

(57) ABSTRACT

There is provided herein an oral care composition comprising porous silica particles having pores in the mesoporous range, wherein the average pore size of the pores in the mesoporous range is from about 7.0 to about 25.0 nm.

### FIELD OF THE INVENTION

The present invention is concerned with oral care compositions containing silica. In particular, the present invention relates to oral care compositions comprising porous silica particles, which compositions are useful in the prevention of tooth decay and the formation of dental caries.

### BACKGROUND TO THE INVENTION

The listing or discussion of any prior-published document in this specification should not necessarily be taken as an acknowledgement that the document is part of the state of the art or is common general knowledge.

Tooth (dental) decay, or the development of dental caries, occurs when the enamel of the tooth is damaged, causing lesions within the tooth which can develop into cavities. Infection of the resulting cavities may lead to complications including inflammation of the tissue around the tooth, tooth loss, infection and abscess formation.

Dental caries may develop for a number of reasons. In particular, they may develop when bacteria in the mouth metabolise sugars to produce acids which demineralize the hard tissues of the tooth (enamel and dentine). Such sugars may be present in the mouth as a direct result of food consumption or may be produced in situ, such as through the action of the salivary enzyme amylase in breaking down starches into sugars (e.g. maltose). Bacteria then engage in the fermentation of such sugars to produce acids (e.g. lactic acid), which in turn cause damage to the tooth enamel.

The reduction of the formation of dental caries is typically achieved through improved oral hygiene, the aim of which is to remove food material from the mouth, and thereby remove sources of starches and sugars, and to reduce the population of oral bacteria, thereby reducing the production of acids through fermentation. Brushing of the teeth with toothpaste products is typically directed at food removal, although some antibacterial effects may also arise. Reduction in bacterial populations may also be achieved through the use of mouthwash products, such as those containing antibacterial agents and/or denaturing agents (e.g. alcohols).

Toothpastes are typically provided in the form of a paste or gel, and will typically contain one or more abrasive agents allowing for removal of food material and dental plaques from the surface of the tooth. Other agents may be included in such products in order to strengthen the enamel of the tooth (e.g. fluoride), to improve flavour, to modify the appearance of the product (e.g. colourants) and to combat halitosis.

Abrasive agents commonly used in toothpaste products include particles of aluminium hydroxide (Al(OH)₃), calcium carbonate (CaCO₃), various calcium hydrogen phosphates, hydroxyapatite (Ca₅(PO₄)₃OH) and silica materials (SiO₂), typically in the form of solid particles thereof.

Porous silica particles have been used in a number of healthcare applications, such as in providing a medium for drug loading and delivery of therapeutic agents. They are thermally and chemically stable, and are exclusively composed of pure silicon dioxide.

These silica particles may possess an ordered porosity with controllable pore dimensions, which gives them a high surface area and large total pore volume. These properties, amongst others such as stability and biocompatibility, make them particularly suited for biomedical applications (see, for example, Wang, Y. et al., Nanomedicine Nanotechnology, Biol. Med. 11, 313-327 (2015)). Moreover, similar materials have previously been approved as food additives (European Center for Ecotoxicology and Toxicology of Chemicals Synthetic Amorphous Silica (CAS No. 7631-86-9), JACC No. 51, page 14 (ECETOC, 2006)).

WO 2014/072363 discusses the use of highly structured, porous silica materials having a specific average pore size of pores in the mesoporous range in the treatment of conditions such as obesity and dyslipidemia. It does not provide any teaching relating to oral hygiene, or the prevention or reduction of dental caries.

### DETAILED DESCRIPTION OF THE INVENTION

We have now found that certain porous silica materials having a specific average pore size of pores in the mesoporous range are able to effectively act as molecular sieves for certain biological molecules in vivo, and thus have properties rendering them useful in oral care, such as in the prevention of or reduction in the formation of dental caries.

Specifically, porous silica particles according to the present invention are designed to possess certain physiochemical properties, as herein described, allowing for a significant biological effect of relevance to the above-mentioned applications. Control of certain particle properties, such as average pore size, has been unexpectedly found to provide these biological effects, such as by allowing for the absorption of salivary amylase enzyme in the mouth, which in turn may lead to reduced production of acids by cariogenic bacteria which are detrimental to oral (e.g. dental) health.

### Oral Care Compositions

In a first aspect of the invention, there is provided an oral care composition comprising porous silica particles having pores in the mesoporous range, wherein the average pore size of the pores in the mesoporous range is from about 7.0 to about 25.0 nm.

For the avoidance of doubt, the silica particles as defined in the first aspect of the invention (including all embodiments and features thereof) may also be referred to as “the silica (or silica material or silica particles) of the invention (or of the first aspect of the invention),” or the like. Similarly, compositions as defined in the first aspect of the invention (including all embodiments and features thereof) may also be referred to as “the composition(s) of the invention (or of the first aspect of the invention),” or the like.

Unless indicated otherwise, all technical and scientific terms used herein will have their common meaning as understood by one of ordinary skill in the art to which this invention pertains.

When used herein in relation to a specific value (such as an amount), the term “about” (or similar terms, such as “approximately”) will be understood as indicating that such values may vary by up to 10% (particularly, up to 5%, such as up to 4%, 3%, 2% or 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

For the avoidance of doubt, the skilled person will understand that where percentages of a certain feature are defined as belonging to different (i.e. non-overlapping) groups, the sum of these percentages cannot exceed 100%. Similarly, where it is possible for such features to belong to other, non-specified groups, there is no requirement for the sum of the specified features to equal 100%.

The skilled person will understand that the porous silica particles as provided in the compositions of the first aspect of the invention may be referred to as a plurality thereof, which plurality may be referred to as a porous silica material.

The skilled person will understand that oral care compositions of the first aspect of the invention include compositions of a general type suitable for cleaning (e.g. by use in brushing, polishing, flushing and/or rinsing) the surfaces of the oral cavity.

As such, the skilled person will understand that references to an oral care composition for the purposes of the present invention may include references to compositions in the form of a paste, powder, liquid, gum, serum or other preparation in a form suitable for cleaning (e.g. by brushing, polishing, flushing, rinsing, or the like) the teeth and/or other surfaces in the oral cavity.

In particular embodiments, the oral care composition of the first aspect of the invention may be delivered in the form of a dentifrice (which may be in a semi-solid form, e.g. a paste, powder or gel) or liquid.

In particular, the skilled person will understand that references to a dentifrice will include references to compositions in an extrudable semi-solid form, such as pastes (i.e. a toothpaste) and gels, and to powders (i.e. a toothpowder), including loose and compressed (e.g. loose) powders, which compositions may be suitable for use in the brushing and/or polishing (e.g. brushing) of the teeth (e.g. using a suitable tooth cleaning implement, such as a toothbrush). As such the term dentifrice may denote an oral composition as described above which is used to clean (the surfaces of) the oral cavity.

The skilled person will also understand that references to gels may refer to substances having the physical properties of a liquid but with substantially zero flow.

In a particular embodiment, the oral care composition may be in the form of a dentifrice, such as a toothpaste or a toothpowder (e.g. a toothpaste), which terms will be known to those skilled in the art.

In such embodiments, the dentifrice may be referred to as being for the brushing and/or polishing (e.g. the brushing) of the surfaces of the oral cavity (e.g. the teeth) in accordance with the typical understanding of users of such products, such as in combination with a suitable cleaning implement (e.g. a toothbrush).

In a particular embodiment, the dentifrice may be in the form of a paste, i.e. a toothpaste (or, similarly, a gel), as known to those skilled in the art. In such embodiments, the composition may comprise, in addition to the porous silica particles as described herein, one or more additional components as typically provided in order to form such compositions, which will include those as described herein.

Thus, in particular embodiments references to an oral care composition may be replaced with references to a toothpaste, which term the skilled person will understand as referring to a composition for use with a suitable tooth cleaning implement such as a toothbrush (e.g. being for cleaning the teeth by brushing in combination with such compositions, such as for a period of about one to two minutes).

In a further embodiment, the composition may be in the form of a liquid, in which case the composition may be in the form of a mouthwash (or mouth/dental rinse). In such embodiments, the composition may comprise, in addition to the porous silica particles as described herein, one or more additional components as typically provided in order to form such compositions, including those as described herein.

Thus, in particular embodiments, references to an oral care composition may be replaced with references to a mouthwash (or mouth rinse, or the like), which term the skilled person will understand as referring to a composition for the rinsing and/or flushing (e.g. rinsing) of the oral cavity (e.g. the surface of the teeth), such as by holding the composition in the mouth for a period of time (e.g. around one minute) and then ejecting (expectorating) the composition from the mouth.

Thus, the particular embodiments of the invention there is provided a mouthwash comprising the silica of the invention.

In further embodiments, the composition may be in the form of a gel, which may be suitable for such uses as described herein in relation to a dentifrice and/or a mouthwash. References to gels may include pastes, mousses, creams, and the like.

It will be understood by those skilled in the art that such compositions are typically not intentionally swallowed for purposes of systemic administration of therapeutic agents, but are instead applied to the oral cavity and then ejected from the body via the mouth (e.g. expectorated). As such, compositions as described herein may be referred to as being non-systemic, topical (as it pertains to the oral cavity, e.g. orally topical), not for swallowing, not for consumption, and the like. Where used in combination with a cleaning implement such as a toothbrush, such compositions may be used in a manner requiring application to the bristles of the toothbrush and then brushing of the accessible surfaces of the oral cavity (e.g. the accessible surfaces of the teeth).

For the avoidance of doubt, the use of mouthwashes and other products as described herein (such as pastes, gels, mousses, creams, etc) may or may not be followed by rinsing of the oral cavity.

As discussed herein, the oral care composition may comprise additional components as typically present in the relevant type of composition, which components will be known to those skilled in the art.

For example, where the oral care composition is provided in the form of a toothpaste, the oral care composition may further comprise components including:
structurants, such as binders and thickening agents, which structurants may be present in an amount of from about 0.1 to 1.0 wt % by weight based on the total weight of the composition. Suitable binders or thickening agents that may be mentioned include carboxyvinyl polymers (such as polyacrylic acids cross-linked with polyallyl sucrose or polyallyl pentaerythritol), hydroxyethyl cellulose, hydroxypropyl cellulose, natural gums (such as carrageenan, gum karaya, guar gum, xanthan gum, gum arabic, and gum tragacanth). Natural gum-based thickeners, in particular carrageenan, may also be mentioned;
an aqueous continuous phase, such as may be formed from a mixture of water and polyhydric alcohol (in various relative amounts), with the amount of water generally ranging from about 10 to about 60 wt % (e.g. about 40%) based on the total weight of the composition and the amount of polyhydric alcohol generally ranging from about 5 to about 70 wt % (e.g. about 30%) of the total weight of the composition. Polyhydric alcohols that may be mentioned include humectants, such as glycerol, sorbitol, polyethylene glycol, polypropylene glycol, propylene glycol, xylitol (and other edible polyhydric alcohols), hydrogenated partially hydrolysed polysaccharides and mixtures thereof. More particular polyhydric alcohols that may be mentioned include glycerol and sorbitol, such as sorbitol. In certain embodiments, the amount of water and/or polyhydric alcohol will generally be at least about 10 wt %, such as at least about 30 wt %, e.g. at least about 50 wt % by total weight water and/or polyhydric alcohol based on the total weight of the composition. In such embodiments, the water and/or polyhydric alcohol may be less than about 90 wt % of the total composition, such as less than about 85 wt %;
abrasive materials, which materials may be present in an amount of from about 0.5 to about 75.0 wt %, such as about about 3.0 to about 60 wt %, based on the total weight of the dentifrice. Particular abrasive material (i.e. abrasive cleaning agents) that may be mention include silica xerogels, hydrogels and aerogels and precipitated particulate silicas (which silica(s) will be present in addition to the porous silica materials as required in the first aspect of the invention), calcium carbonate, dicalcium phosphate, tricalcium phosphate, calcined alumina, sodium and potassium metaphosphate, sodium and potassium pyrophosphates, sodium trimetaphosphate, sodium hexametaphosphate, particulate hydroxyapatite and mixtures thereof. For the avoidance of doubt, the skilled person will understand that the essential silica component of the first aspect of the invention may also function as an abrasive agent;
a thickener and/or gelling agent (in each case, inorganic, natural or synthetic), which agents may be present in amounts from about 0.1 to about 15.0 wt % of the total composition. The skilled person will understand that the amount (and proportions) of thickener(s) will be selected in order to form an extrudable, shape-retaining product which can be squeezed from a tube onto a toothbrush and will not fall between the bristles of a toothbrush but, rather, will substantially maintain its shape thereon. Particular thickeners and gelling agents that may be mentioned include finely divided silicas (which silica(s) will be present in addition to the porous silica materials as required in the first aspect of the invention), hectorites, calcium carbonate, colloidal magnesium aluminium silicates and mixtures thereof and/or gums such as Irish moss, iota-carrageenan, gum tragacanth, and polyvinylpyrrolidone. For the avoidance of doubt, the skilled person will understand that the essential silica component of the first aspect of the invention may also function as a thickener;
metal ions, such as tin or zinc (e.g. zinc). Particular metal ions that may be mentioned include zinc ions are zinc chloride, zinc acetate, zinc gluconate, zinc sulphate, zinc fluoride, zinc citrate, zinc lactate, zinc oxide, zinc monoglycerolate, zinc tartrate, zinc pyrophosphate zinc maleate and mixtures thereof;
fluoride sources. Particular fluoride sources that may be mentioned include sodium fluoride, stannous fluoride, sodium monofluorophosphate, zinc ammonium fluoride, tin ammonium fluoride, calcium fluoride, cobalt ammonium fluoride and mixtures thereof; and
further optional ingredients customary in the art, such as antimicrobial agents such as chlorhexidine, sanguinarine extract, metronidazole, quaternary ammonium compounds (e.g. cetylpyridinium chloride), bis-guanides (e.g. chlorhexidine digluconate, hexetidine, octenidine and alexidine) and halogenated bisphenolic compounds (e.g. 2,2′methylenebis-(4-chloro-6-bromophenol)), anti-inflammatory agents such as ibuprofen, flurbiprofen, aspirin and indomethacin, anti-caries agents such as sodium-and stannous fluoride, aminefluorides, sodium monofluorophosphate, sodium trimeta phosphate and casein, plaque buffers such as urea, calcium lactate, calcium glycerophosphate and strontium polyacrylates, vitamins such as Vitamins A, C and E, plant extracts, plant-derivable antioxidants such as flavonoid, catechin, polyphenol, and tannin compounds and mixtures thereof, desensitising agents such as potassium citrate, potassium chloride, potassium tartrate, potassium bicarbonate, potassium oxalate, potassium nitrate and strontium salts, anti-calculus agents such as alkali-metal pyrophosphates, hypophosphite-containing polymers, organic phosphonates and phosphocitrates, biomolecules such as bacteriocins, antibodies and enzymes, flavours such as peppermint and spearmint oils, proteinaceous materials such as collagen, preservatives, opacifying agents, amino acids such as arginine, colouring agents, sweetening agents, pharmaceutically acceptable carriers such as starch, sucrose (in addition to water or water/alcohol systems as referred to above), pH-adjusting agents including buffers and salts to buffer the pH and ionic strength of the oral care composition, bleaching agents such as peroxy compounds (e.g. potassium peroxydiphosphate), effervescing systems such as sodium bicarbonate/citric acid systems, and colour change systems.

Similarly, where the oral care composition is provided in the form of a liquid (e.g. a mouthwash), the oral care composition may further comprise components including water, emollients (e.g. glycerol), sweeteners (e.g. xylitol), preservatives (e.g. sodium benzoate) and/or sodium fluoride.

The skilled person will also understand that such mouthwash formulations may comprise further ingredients as may be desirable in the circumstances, such as anti-bacterial agents (e.g. an alcohol), colourants and/or flavourings (such as menthol).

Without wishing to be bound by theory, it is thought that the presence of surfactants, such as those commonly used in oral care compositions (e.g. toothpastes), may inhibit or reduce the effects of the silica particles and thus reduce the beneficial effects of the composition.

Thus, in particular embodiments, the oral care composition (e.g. the dentifrices, such as a toothpaste or mouthwash) is substantially free of surfactant.

Particular surfactants that may be mentioned include:
anionic surfactants, such as sodium lauryl sulfate;
zwitterionic (zero net charge) surfactants, such as cocamidopropyl betaine; and
nonionic surfactants, such as polyethoxylated fatty acid sorbitan esters (e.g. polysorbate 80), ethoxylated fatty acids, esters of polyethylene glycol, ethoxylates of fatty acid, monoglycerides and diglycerides, and ethylene oxide/propylene oxide block polymers, and polyethylene glycol ethers of fatty alcohols, in particular polyethylene glycol ethers having from 2 to 200 (e.g. 20 to 40) ethylene oxide groups per unit, such as polyoxyethylene (2-100, e.g. 4) lauryl ether, and ‘steareth’ surfactants, such as Steareth 30.

As used herein, the skilled person will understand that the reference to being substantially free of surfactant may refer to presence of components classified as surfactants (such as those referred to herein) at concentrations at or below about 0.5 wt % of the total composition, such as at or below about 0.4, 0.3, 0.02 or, particularly, 0.1 wt % of the total composition (e.g. at or below about 0.09, 0.08, 0.07, 0;06 or 0.05 wt % of the total composition, particularly below about 0.04, 0.03, 0.02 or 0.01 wt % of the total composition).

In particular embodiments, the skilled person will understand that the reference to being substantially free of surfactant may refer to the absence of (i.e. the absence of detectable levels of) components classified as surfactants, which may indicate that the preparation of such compositions does not involve addition of any such components.

As used herein, being substantially free of a component may be indicated by stating that the composition “does not contain a substantial concentration of” or “does not contain” that component, respectively.

The skilled person will also understand that the maximum about of surfactant that may be present may vary depending on the nature of the surfactant component, with such levels being determined using routine techniques.

For example:
in respect of anionic surfactants, such as sodium lauryl sulfate, the surfactant may be present at levels at or below about 0.05 wt % (e.g. below about 0.04, 0.03, 0.02 or, particularly, 0.01 wt %), or may be referred to in terms of the absence thereof;
in respect of zwitterionic (zero net charge) surfactants, such as cocamidopropyl betaine, the surfactant may be present at levels at or below about 0.05 wt %;
nonionic surfactants, such as polyethoxylated fatty acid sorbitan esters (e.g. polysorbate 80), the surfactant may be present at levels at or below about 0.1 (or, particularly, 0.05) wt %, or may be referred to in terms of the absence thereof.

In particular embodiments, where amounts of specific surfactants are referred to, compositions will be substantially free of other surfactants.

The skilled person will understand that the oral care composition may be provided in the form of a mixture of the various components thereof. In particular, the skilled person will understand that such mixtures may comprise components in both the liquid (or gel) and solid phases (e.g. as solid particles), in which case in use the solid component(s) may be substantially evenly distributed through the liquid (or gel) components, which in the case of liquid compositions may require the composition to be agitated (e.g. shaken) before use.

For the avoidance of doubt, in the case of liquid compositions (e.g. a mouthwash), the silica particles may be heterogenous with the liquid composition, resulting in sedimentation of the particles during storage. Thus, use of the composition may require mixing (e.g. by shaking and/or inversion thereof) prior to use.

For the avoidance of doubt, the skilled person will understand that references herein to particles forming part of a composition may include only particles of a suitable size to be considered as forming part of the composition (i.e. particles that may be able to function as a component of the composition).

The skilled person will be able to determine the amount of the silica of the invention required in compositions of the invention in order to provide the effects as described herein, which amounts may depend on the type of composition used.

In particular embodiments, the silica of the invention may be present in compositions of the invention in amounts from about 0.1 to about 20.0 wt %.

For example, the silica of the invention may be present in compositions of the invention in amounts of about 0.5 wt % (e.g. about 0.44 wt %).

As used herein, the term “consists essentially of” may indicate that the relevant composition consists of at least 90% by weight (e.g. at least 95% by weight, such as at least 99% by weight or, particularly, at least 99.9%) of the relevant substance.

In certain embodiments of first aspect of the invention, such as wherein the composition is in the form of a dental powder, the composition consists (or consists essentially of) the porous silica particles as defined herein (i.e. a plurality of such particles).

In alternative embodiments of the first aspect of the invention, the porous silica particle content (or, alternatively, the silica particle content) of the composition consists of (or consists essentially of) the silica particles as defined herein (i.e. such that components other than porous silica material may be present).

The skilled person will understand that the properties of the silica of the invention may be such that the use of other enzyme inhibiting/denaturing and/or adsorbing materials/substances is not required (i.e. the composition of the invention may produce the effects as described herein without the need for the presence of such agents).

In particular embodiments of first aspect of the invention, the composition comprises the porous silica material (as defined in the first aspect of the invention) as the only (i.e. sole) ingredient capable of adsorbing enzymes.

Thus, in further embodiments of first aspects of the invention, the composition is substantially free of other enzyme adsorbing ingredients.

As used in relation to other enzyme adsorbing ingredients, the term substantially free will refer to the essential material (e.g. the composition referred to) comprising no significant (i.e. clinically significant) amount of the other material referred to (e.g. the other therapeutically active ingredient(s)), which may indicate the presence of less than 10% (e.g. less than 5%, such as less than 2%, less than 1%, less than 0.5% or, particularly, less than 0.1%, less than 0.01% or less than 0.001%) by weight of the other material, or more particularly the presence of no detectable amount of the other material.

### Porous Silica Particles

The skilled person will understand that references herein to pores being of a certain size will refer to the average diameter of the relevant pores (i.e. the average diameter of each individual pore, considering the dimensions thereof). For the avoidance of doubt, the skilled person will understand that references to average pore size may refer in particular to the average size of the opening of each pore (or, in the case of a pore the channel of which internally traverses the body of the particle, the average size of all openings to the pore(s)), which may be referred to as the pore window(s) (or the window(s) of the pore).

For the avoidance of doubt, unless otherwise stated, averages referred to herein will be calculated as the mean average.

Unless otherwise stated, pore sizes as described herein is measured by nitrogen sorption and calculated using the Density Functional Theory (DFT) method (see, for example, the methods as described in Landers, J. et al., Colloids and Surfaces A: Physicochem. Engineering Aspects, 437, 3-32 (2013)). As such, unless otherwise stated, references herein to an average pore size will refer to pore size as measured by nitrogen sorption and calculated using the Density Functional Theory (DFT).

Alternatively, pore size may be measured by nitrogen sorption and calculated using the Barrett-Joyner-Halenda (BJH) model (see Barrett, E. P.; Joyner, L. S.; and Halenda, P. P., J. Am. Chem. Soc. 73, 373-380 (1951)), with any pore size measurements herein calculated in this way being indicated as such.

The skilled person will understand that references to the percentage of pores present being in a particular range may be understood to be references to the pore size distribution (PSD) of such particles. As such, references to the percentage of pores present being in a particular range will refer to the combined volume of pores present in each range as a percentage of the total pore volume of the relevant group(s) of pores (e.g. pores in the mesoporous range).

For the avoidance of doubt, references to particles having a particular average pore size may in certain instances include references to pores that are functionally equivalent (e.g. when utilised in the manner described herein) with particles having such average pore sizes.

The skilled person will understand that pore size distribution of the silica material may be measured using DFT pore size distribution curves, which is a technique well-understood by those skilled in the art (see, for example, Olivier, J. P., Conklin, W. B. and Szombathely, M. V., Studies in Surface Science and Catalysis, 87, 81-89 (1994)). The percentage of the pores are calculated from the DFT cumulative pore size distribution curves.

The skilled person will understand that references to porous silica particles having pores in the mesoporous range will take its normal meaning in the art, i.e. as referring to porous silica particles having (or containing/comprising) pores with a diameter in the range 2 to 50 nm, which materials may be referred to as mesoporous and which pores may be referred to as mesopores.

For the avoidance of doubt, the skilled person will understand that the porous silica material referred to in the first aspect of the invention may also have (i.e. further containing/comprising) pores with a diameter outside of the mesoporous range, such as by having micropores (i.e. pores with a diameter of less than 2 nm) and/or macropores (i.e. pores with a diameter of greater than 50 nm).

For the avoidance of doubt, unless otherwise stated, references for percentages of pores as used herein will refer to the percentage by volume.

In a particular embodiment, at least about 40% (i.e., 40% by volume) of the pores present in the silica material of the invention are in the mesoporous range.

In a more particular embodiment, at least about 50%, such as at least about 60%, particularly at least about 70%, of the pores present in the silica material of the invention are in the mesoporous range.

The skilled person will understand that, in relation to the pores in a given range, there may also be calculated an average (i.e. mean average) pore size. As described herein, such average pore size may be measured by the nitrogen sorption technique and calculated using the Density Functional Theory (DFT), which will be well-known to those skilled in the art (see: Olivier, J. P., Conklin, W. B. and Szombathely, M. V., Studies in Surface Science and Catalysis, 87, 81-89 (1994); Landers, J., et al., Colloids and Surfaces A: Physicochem. Eng. Aspects, 437, 3-32 (2013)). As such, unless otherwise stated, references herein to average pore size will refer to average pore size as measured by nitrogen sorption and calculated using DFT.

In particular embodiments, the average pore size of the pores in the mesoporous range is from about 7.0 to about 22.0 nm.

In more particular embodiments, the average pore size of the pores in the mesoporous range is from about 7.0 to about 21.0 nm.

In yet more particular embodiments, the average pore size of the pores in the mesoporous range is from about 7.0 to about 20.0 nm.

For example, in certain embodiments the average pore size of the pores in the mesoporous range is:
from about 7.0 to about 19.0 nm;
from about 7.0 to about 18.0 nm;
from about 7.0 to about 17.0 nm;
from about 7.0 to about 16.0 nm;
from about 7.0 to about 15.0 nm;
from about 7.0 to about 14.0 nm;
from about 7.0 to about 13.0 nm; or
from about 7.0 to about 12.0 nm.

In certain embodiments, the average pore size of the pores in the mesoporous range is from about 8.0 to about 13.0 nm.

In more particular embodiments, the average pore size of the pores in the mesoporous range is from about 8.0 to about 12.0 nm.

In more particular embodiments, the average pore size of the pores in the mesoporous range is from about 8.0 to about 11.0 nm.

In alternative embodiments, the average pore size of the pores in the mesoporous range is from about 9.0 to about 11.0 nm.

In yet more particular embodiments, the average pore size of the pores in the mesoporous range is from about 9.2 to about 11.0 nm.

In yet more particular embodiments, the average pore size of the pores in the mesoporous range is from about 9.4 to about 10.8 nm.

In yet more particular embodiments, the average pore size of the pores in the mesoporous range is from about 9.5 to about 10.7 nm.

In yet more particular embodiments the average pore size of the pores in the mesoporous range is from about 9.6 to about 10.7 nm.

In yet more particular embodiments, the average pore size of the pores in the mesoporous range is from about 9.5 to about 10.6 nm.

In the most particular embodiments, the average pore size of the pores in the mesoporous range is from about 9.6 to about 10.6 nm.

The skilled person will understand that, in addition to referring to the (mean) average pore size, as described herein, the silica material of the invention may also be defined by reference to the distribution of pore sizes, such as the distribution of pore sizes of the pores in the mesoporous range.

In particular embodiments of the first aspect of the invention, at least 21% (such as at least at least 22%, at least 23%, at least 24%, at least 25%, at least 26%, at least 27% at least 28% or at least 29%) of the pores in the mesoporous range (by volume) have a diameter in within the range of the specified for the average pore size (i.e. the range as specified for the average pore size).

In more particular embodiments of the first aspect of the invention, at least about 30% of the pores in the mesoporous range have a diameter in within the range of the average pore size.

In yet more particular embodiments of the first aspect of the invention, at least about 35% (such as at least 40% or at least 45%) of the pores in the mesoporous range have a diameter in within the range of the average pore size.

In still more particular embodiments of the first aspect of the invention, at least about 50% (such as at least 55%, at least 60%, at least about 65%, at least about 70% or, particularly, at least about 72%) of the pores in the mesoporous range have a diameter in within the range of the average pore size (i.e. the range given for the average pore size of the pores in the mesoporous range, as defined herein).

For example, in certain embodiments at least about 50% (such as at least 55%, at least 60%, at least about 65%, at least about 70% or, particularly, at least about 72%) of the pores in the mesoporous range have a diameter in within the range about 7.0 to about 25.0 nm.

In certain embodiments, up to about 100% (or up to about 99%, about 95%, or about 90%) of the pores in the mesoporous range have a diameter in within the range of the average pore size (i.e. the average pore size range as specified herein).

In certain embodiments, from about 21% to about 100% (or, particularly, about 25% to about 99% or 100%) of the pores in the mesoporous range have a diameter in within the range of the average pore size.

In yet more particular embodiments of the first aspect of the invention, at least about 30% (e.g. about 30% to about 99% or 100%) of the pores in the mesoporous range have a diameter in within the range of the average pore size.

In yet more particular embodiments of the first aspect of the invention, at least about 35% (e.g. about 35% to about 99%) of the pores in the mesoporous range have a diameter in within the range of the average pore size.

In yet more particular embodiments of the first aspect of the invention, about 40% to about 90% (or to about 99% or 100%) of the pores in the mesoporous range have a diameter in within the range of the average pore size.

In certain embodiments of the first aspect of the invention, about 50% to about 90% (or to about 99% or 100%) of the pores in the mesoporous range have a diameter in within the range of the average pore size.

In certain embodiments of the first aspect of the invention, about 55% to about 90% (or to about 99% or 100%) of the pores in the mesoporous range have a diameter in within the range of the average pore size.

In certain embodiments of the first aspect of the invention, about 60% to about 90% (or to about 99% or 100%) of the pores in the mesoporous range have a diameter in within the range of the average pore size.

For example, in a particular embodiment (i.e. a particular embodiment of the first aspect of the invention), at least about 25% (e.g. about 25% to about 99%, such as about 50% to about 99% or 100%) of the pores of the silica particle are mesopores of a size in the range of from about 7.0 to about 25.0 nm (such as about 7.0 to about 18.0 nm, or about 7.0 to about 13.0 nm).

Similarly, in a particular embodiment, at least about 50% (e.g. about 50% to about 99%, such as about 50% to about 90%) of the pores of the silica particle are mesopores of a size in the range of from about 7.0 to about 25.0 nm (such as about 7.0 to about 18.0 nm, or about 7.0 to about 13.0 nm).

In a particular embodiment (i.e. a particular embodiment of the first aspect of the invention), at least about 50% (e.g. about 50% to about 99%) of the pores of the silica particle are mesopores of a size in the range of from about 9.0 to about 12.0 nm.

In a further embodiment, at least about 25% (e.g. at least about 50%, about 60% or about 70%) of the pores of the silica particle are mesopores of a size in the range of from about 9.0 to about 10.2 nm.

In a further embodiment, at least about 25% (e.g. at least about 50%, about 55%, about 60%, about 65% or about 70%) of the pores of the silica particle are mesopores of a size in the range of from about 9.0 to about 11.0 nm.

In a more particular embodiment, at least about 25% (e.g. about 25% to about 99%) of the pores of the silica particle are mesopores of a size in the range of from about 9.0 to about 11.0 nm.

In a yet more particular embodiment, at least about 25% (e.g. about 25% to about 99%) of the pores of the silica particle are mesopores of a size in the range of from about 9.0 to about 10.2 nm.

The skilled person will understand that references to porous silica particles having pores in the mesoporous range will necessarily require that such particles are porous, which will include particles behaving in a porous manner. As such, porous silica particles will refer to particles having a significant degree of porosity, which may in certain embodiments be defined by reference to features such as the pore volume and/or surface area of the particles, such as by reference to those parameters as defined herein (which features as described herein may, as with other features described herein, be taken both alone and in combination).

The skilled person will also understand that the total volume of pores in each particle may affect the surface area of the particle. Thus, the preparation of a particle with a greater pore volume may allow for, and be defined in terms of, a greater particle surface area.

The skilled person will understand that the surface area of a particle (or a sample of particles) may be calculated using the Brunauer Emmett Teller (BET) theory, a technique well-known to those skilled in the art (see, for example, Brunauer, S., Emmett, P. H., and Teller, E., J. Am. Chem. Soc., 60(2), 309-319 (1938)).

In a particular embodiment, the silica particles have a BET surface area of at least about 150 m₂/g.

In a more particular embodiment, the silica particles have a BET surface area of at least about 200 m₂/g.

In a yet more particular embodiment, the silica particles have a BET surface area of at least about 300 m₂/g (such as at least about 350 m₂/g).

In a still more particular embodiment, the silica particles have a BET surface area of at least about 400 m₂/g (such as at least about 450 m₂/g).

In a particular embodiment, the silica particles have a BET surface area of at least about 500 m₂/g.

In particular embodiments, the BET surface area is up to about 1500 m₂/g (such as up to about 1200 m₂/g or 1000 m₂/g).

For example, in a particular embodiment, the silica particles have a BET surface area of from about 200 to about 1500 m₂/g.

In a further embodiment, the silica particles have a BET surface area of from about 500 to about 1200 m₂/g.

In a yet more particular embodiment, the silica particles have a BET surface area of from about 600 to about 1200 m₂/g.

In an alternative embodiment, the silica particles have a BET surface area of from about 600 to about 1000 m₂/g.

In a further alternative embodiment, the silica particles have a BET surface area of from about 500 to about 900 m₂/g, such as from about 550 to about 900 m₂/g.

In a yet further alternative embodiment, the silica particles have a BET surface area of from about 600 to about 850 m₂/g.

The skilled person will understand that the porous silica particles may be provided in a variety of shapes.

In a particular embodiment, the silica particles have a substantially non-spherical morphology (i.e. an aspect ratio of greater than 1:1, such as greater than 1.1:1).

In a more particular embodiment, the silica particles have an aspect ratio of greater than 1.5:1, such as greater than 1.8:1.

In a yet more particular embodiment, the silica particles have an aspect ratio equal to or greater than 2:1.

As used herein, the term “aspect ratio” will be understood to refer to the ratio between the largest cross-section diameter of the silica particle and the smallest cross-section diameter.

Alternatively, such particles (i.e. particles having a substantially non-spherical morphology) may be described as having at least one plane (i.e. an equally dividing plane bisecting the particle) of asymmetry (i.e. such that the morphology of the particle differs about the plane).

In a more particular embodiment, the silica particles have an essentially rod-shaped morphology. Thus, in particular embodiments, the porous silica particle may be characterized by having an essentially rod-shaped morphology, as seen by electron microscopy (such as by Scanning Electron Microscopy (SEM) or Transmission Electron Microscopy (TEM), using techniques known to those skilled in the art), such as with a rod-length of from about 0.5 to about 5.0 μm.

As used herein, the term essentially rod-shaped will be understood as referring to a particle of an elongate form resembling a rod, in which the rod may be straight or curved (e.g. such rod shaped particles may be substantially straight).

In an alternative embodiment, the silica particles of the invention may be substantially spherical (or referred to as spherical). Thus, in a particular embodiment, the silica particles of the invention may have an aspect ratio (or an average aspect ratio) of about 1:1.

In further embodiments, the silica particles of the invention may be of amorphous shape.

The skilled person will understand that the term mean particle size, as used herein, will refer to the mean diameter of the particles at the greatest point thereof (e.g. in the case of rod-shaped particles, the length thereof; or in the case spherical particles, the diameter thereof.), which may be measured using techniques well-known to those skilled in the art, for example using electron microscopy techniques (such as by Scanning Electron Microscopy (SEM) or Transmission Electron Microscopy (TEM) technique known to those skilled in the art). In a particular embodiment, particle size is determined using electron microscopy (e.g. using SEM).

In particular embodiments, such as those in which the particles are spherical, the size of particles may be defined by reference to the diameter thereof.

In a particular such embodiment, the silica particles have a mean particle size of from about 0.1 to about 20.0 μm.

In a more particular embodiment, the silica particles have a mean particle size of from about 0.1 to about 15.0 μm.

In a yet more particular embodiment, the silica particles have a mean particle size of from about 0.1 to about 10.0 μm.

In a yet more particular embodiment, the silica particles have a mean particle size of from about 0.5 to about 10.0 μm.

In a still more particular embodiment, the silica particles have a mean particle size of from about 0.5 to about 5.0 μm.

In certain embodiments, the silica particles have a mean particle size of from about 0.5 to about 4.5 μm.

In particular embodiments, the silica particles have a mean particle size of from about 1.0 to about 10.0 μm.

In particular embodiments, the silica particles have a mean particle size of from about 1.0 to about 5.0 μm.

In more particular embodiments, the silica particles have a mean particle size of from about 1.0 to about 4.0 μm.

In more particular embodiments, the silica particles have a mean particle size of from about 1.0 to about 4.0 μm.

In yet more particular embodiments, the silica particles have a mean particle size of from about 2.0 to about 4.0 μm.

In still more particular embodiments, the silica particles have a mean particle size of from about 3.0 to about 4.0 μm.

In further embodiments, such as those in which the particles are rod-shaped, the size of particles may be defined (or also defined) by reference to the width thereof (which will refer to the diameter at the narrowest point).

In a particular such embodiment, the silica particles have a mean width of from about 0.05 to about 0.6 μm.

In a more particular embodiment, the silica particles have a mean width of from about 0.1 to about 0.6 μm.

In a yet more particular embodiment, the silica particles have a mean width of from about 0.1 to about 0.4 μm.

In a yet more particular embodiment, the silica particles have a mean width of from about 0.2 to about 0.4 μm.

The skilled person will understand that porous silica materials of the type described in the present invention are typically non-crystalline. Thus, in certain embodiments, the porous silica particle may be described as a substantially non-crystalline porous silica particle (and materials formed from a plurality of such particles may be described in the same manner). As such, the porous silica particle may be described as a non-crystalline porous silica particle.

In alternative embodiments, the silica material present in particles as described in the first aspect of the invention may be described as being amorphous. In such embodiments, it will be understood that the term amorphous will indicate that the structure of the silica material (excluding the pores present therein) has no substantial order, such as the order which may be present in a crystalline substance (i.e. the porous silica particles, or silica material, may be referred to as non-crystalline).

As described herein, the skilled person will understand that the silica materials of the invention are porous. As such, silica particles of the invention may be referred to as having a certain minimum total pore volume, as measured using nitrogen sorption (e.g. taken as the volume adsorbed at the highest value of P/P₀, for example, P/P₀=0.995), or a range of such volumes.

In particular embodiments, the total pore volume is at least about 0.2 cm³/g (such as at least about 0.3, 0.4, 0.5, 0.6 or 0.7 cm³/g).

In particular embodiments, the total pore volume is from about 0.2 to about 2.5 cm³/g.

In more particular embodiments, the total pore volume is from about 0.2 to about 2.0 cm³/g.

In yet more particular embodiments, the total pore volume is from about 0.5 to about 1.5 cm³/g.

In still more particular embodiments, the total pore volume is from about 0.6 to about 1.4 cm³/g.

For example, in certain embodiments, the total pore volume is from about 0.7 to about 1.3 cm³/g.

### Uses and Processes

As described herein, the oral care composition of the first aspect of the invention may be useful in oral care, such as in the prevention (or prophylaxis) of dental caries, the accumulation of dental plaque, gum disease, periodontitis, and/or tooth loss in a subject in need thereof.

In some embodiments, the oral care composition of the first aspect of the invention may be useful in oral care, such as in the prevention (or prophylaxis) of dental caries, the accumulation of dental plaque, gum disease, and/or tooth loss in a subject in need thereof.

In a second aspect of the invention, there is provided the use of an oral care composition as defined in the first aspect of the invention in the prevention (or prophylaxis) of dental caries, the accumulation of dental plaque, gum disease, periodontitis, and/or tooth loss.

In some embodiments, the use of an oral care composition is in the prevention (or prophylaxis) of dental caries, the accumulation of dental plaque, gum disease, and/or tooth loss.

In an alternative second aspect of the invention, there is provided an oral care composition as defined in the first aspect of the invention for use in the prevention (or prophylaxis) of dental caries, the accumulation of dental plaque, gum disease, periodontitis, and/or tooth loss.

In some embodiments, the oral care composition is for use in the prevention (or prophylaxis) of dental caries, the accumulation of dental plaque, gum disease, and/or tooth loss.

In a further alternative second aspect of the invention, there is provided a method of preventing (or prophylaxis of) the formation of dental caries, the accumulation of dental plaque, gum disease, periodontitis, and/or tooth loss, in a subject in need thereof, comprising the step of using (or administering/applying, such as to oral cavity, i.e. the mouth, e.g. the surfaces of the teeth and gums) an effective amount of an oral care composition as defined in the first aspect of the invention.

In some embodiments, there is provided a method of preventing (or prophylaxis of) the formation of dental caries, the accumulation of dental plaque, gum disease, and/or tooth loss, in a subject in need thereof, comprising the step of using (or administering/applying, such as to oral cavity, i.e. the mouth, e.g. the surfaces of the teeth and gums) an effective amount of an oral care composition as defined in the first aspect of the invention.

As used herein, the term prevention (and, similarly, preventing) will include references to the prophylaxis of a condition (and vice-versa). In particular, such terms term may refer to achieving a reduction (for example, at least a 10% reduction, such as at least a 20%, 30% or 40% reduction, e.g. at least a 50% reduction) in the likelihood of a subject developing the condition.

For the avoidance of doubt, the skilled person will understand that such uses and methods will be performed in a subject in need thereof. The need of a subject for such uses and methods may be assessed by those skilled the art using routine techniques.

As used herein, references to a subject will refer to a living subject being treated, including mammalian (e.g. human) patients. In particular, references to a subject will refer to human, such as a human of adult age (i.e. a human aged 18 years or over).

The skilled person will understand that uses and methods relating to the oral care composition of the first aspect of the invention may further comprises such steps as may be appropriate for its use in the form provided.

For example, where the oral care composition is provided in the form of a dentifrice, such uses and methods may comprise the step(s) of using the composition to brush or polish (i.e. clean, e.g. by brushing) the teeth, such as through the application of the composition to a suitable cleaning implement (e.g. a toothbrush) followed by the cleaning (brushing or polishing, such as brushing) of the teeth using the same.

Similarly, where the oral care composition is provided in the form of a liquid (e.g. a mouthwash), such uses and methods may comprise the step(s) of using the composition to rinse or flush (e.g. rinse) the mouth, such as by taking a suitable amount of the composition into the mouth, holding in the mouth (and optionally moving around the mouth) for a period of time (e.g. about 30 to about 60 seconds) and then ejecting the composition from the mouth.

As described herein, compositions of the invention may also take the form of powders or gels, such as a mousse, which may be used in accordance with the common usage of such products as known in the art. For example, when in the form of a mousse, compositions may be applied to the oral cavity for a period of time (such as about one minute), following which the oral cavity may or may not be rinsed with water (e.g. the oral cavity is not rinsed, in which case the composition may be referred to as non-rinse).

As described herein, compositions as described in the first aspect of the invention may be used generally in oral care, such as in the cleaning of the oral cavity (e.g. the surfaces of the teeth, such as by brushing, polishing, rinsing, flushing, or the like).

In a third aspect of the invention, there is provided the use of a composition as described in the first aspect of the invention as an oral care product.

The skilled person will understand that references to oral care products will include references to oral health products (i.e. products for promoting oral health), such as products for the prevention or prophylaxis of the accumulation of dental plaque, gum disease, periodontitis, and/or tooth loss, and for the treatment or prevention (or prophylaxis) of infections of the mouth.

In a fourth aspect of the invention, there is provided the use of a composition as described in the first aspect of the invention in a method of cleaning (e.g. by brushing, polishing, rinsing or flushing) the oral cavity (e.g. the teeth, such as the surfaces thereof).

The skilled person will understand that methods of cleaning the oral cavity (i.e. the inside of the mouth, such as the surfaces of the teeth and gums), as described herein, may comprise the step of applying the composition as described in the first aspect of the invention to the oral cavity.

The skilled person will understand that the oral care composition as described in the first aspect of the invention may be prepared using standard techniques as known in the art, such as through mixing of the components thereof.

Thus, in a further aspect of the invention there is provided a process for preparing an oral care composition comprising the bringing the components of the composition in the form of a mixture (such as a substantially homogenous mixture) thereof.

In a still further aspect of the invention, there is provided the use of a silica material as defined in relation to the first aspect of the invention in the manufacture of a composition as defined herein (e.g. in the first and second aspects of the invention).

Without wishing to be bound by theory, it is believed that the use of certain porous silica materials having a specific average pore size of pores in the mesoporous range that are able to effectively act as molecular sieves for biological molecules in vivo allows for the preparation of oral care compositions having improved properties in the reduction of the formation of dental caries and in other aspects of improved oral hygiene.

In particular, it is believed that the use of mesoporous silica particles having a specific average pore size of pores in the mesoporous range, as described herein, allows for the effective absorption of salivary amylase enzyme in the mouth, which is not observed for silica particles lacking such pores and so provides advantages over oral care compositions as known in the art. Further, the use of such mesoporous silica particles is believed to reduce cariogenic bacterial biofilm formation, which has further benefits in improving oral health.

### BRIEF DESCRIPTION OF THE FIGURES

FIGS. 1.1(A)-(D): Nitrogen sorption analysis. Adsorption-desorption isotherms (FIGS. 1.1(A) and 1.1(B)) and pore size distributions derived from the adsorption curve using the DFT model (FIGS. 1.1(C) and 1.1(D)) of the different types of silicas described in Example 1.

FIG. 1.2: SEM micrographs of silicas from Example 1

FIG. 2.1: Silica in toothpaste effectively reduces carbohydrate digestion. Various types of silicas were formulated in toothpaste without surfactant (TP1) and tested for their efficacy in reducing carbohydrate digestion by human salivary amylase. The same toothpaste formulation without any silica (No silica) was included as a control, which represented the basal digestion level (dotted line). Data are presented as mean±Standard Error (SE) (n=2).

FIG. 2.2: Silica in mouthwash formulation without surfactant effectively reduces carbohydrate digestion. Various types of silicas were formulated in mouthwash without surfactant (MW1) and tested for their efficacy in reducing carbohydrate digestion by human salivary amylase. The same mouthwash formulation without any silica (No silica) was included as a control, which represented the basal digestion level (dotted line). Data are presented as mean±SE (n=2).

FIG. 2.3: Mouthwash containing surfactant diminishes silica's efficacy in reducing carbohydrate digestion. Various types of silicas were prepared in mouthwash formulations containing either polysorbate 80 (A, MW2) or PEG-40 castor oil (B, MW3) and tested for their efficacy in reducing carbohydrate digestion by human salivary amylase. Data are presented as mean±SE (n=2).

FIG. 2.4: Effect of cocamidopropyl betaine in carbohydrate digestion assay. Silica 3 was suspended in different concentrations of the cocamidopropyl betaine solution (surfactant) and its efficacy in reducing carbohydrate digestion by human salivary amylase was assessed. The same concentrations of cocamidopropyl betaine solutions without silica (No silica) were also assessed to obtain the basal level of digestion at those concentrations. Data are presented as mean±SE (n=2).

FIG. 2.5: Effect of Polysorbate 80 in carbohydrate digestion assay. Silica 3 was suspended in different concentrations of the Polysorbate 80 solution (surfactant) and its efficacy in reducing carbohydrate digestion by human salivary amylase was assessed. The same concentrations of Polysorbate 80 solutions without silica (No silica) were also assessed to obtain the basal level the digestion at those concentrations. Data are presented as mean±SE (n=2).

FIG. 2.6: Effect of polyoxyethylene (4) lauryl ether in carbohydrate digestion assay. Silica 3 was suspended in different concentrations of the polyoxyethylene (4) lauryl ether solution (surfactant) and its efficacy in reducing carbohydrate digestion by human salivary amylase was assessed. The same concentrations of polyoxyethylene (4) lauryl ether solutions without silica (No silica) were also assessed to obtain the basal level of digestion at those concentrations. Data are presented as mean±SE (n=2).

FIG. 2.7: Silica with a similar average pore size as silica of the invention but with broad pore size distribution (silica 8) was not able to reduce carbohydrate digestion. ‘No silica’ sample represents the basal level of digestion, and the level of digestion for silica 8 does not differ from the basal level. Data are presented as mean±SE (n=2).

FIG. 3: Silica reduces carbohydrate digestion by salivary amylase in human saliva. Two different types of silica were incubated with human saliva to determine their efficacies in reducing carbohydrate digestion. Data are presented as mean±Standard Deviation (SD) (n=4).

FIGS. 4.1(A)-(E): Effect of different silicas on cariogenic bacterial growth. Silica 3 and 4 were pre-incubated with human salivary amylase and subsequently subjected to carbohydrate digestion assay. After enzyme de-activation, digested products of starch were fed to S. mutans cultures. Several measurements were taken: bacterial growth for the first 8 hr (A, B), amount of undigested starch in the digestion mix after salivary amylase de-activation at various time points (C), bacterial mass 24 hr after the exposure to the digested products (D), and amount of undigested starch in the culture medium 24 hr after the exposure to the digested products (E). Data are presented as mean±SE (n=28).

FIGS. 4.2(A)-(E): Effect of different silicas on cariogenic bacterial growth in 1% starch medium. Silica 3 and 4 were pre-incubated with human salivary amylase. The pre-incubation mix was added to S. mutans culture containing 1% starch. Several measurements were taken: bacterial growth (A, B), amount of undigested starch in the culture medium after the exposure to the pre-incubation mix at various time points (C), bacterial mass 24 hr after the exposure to the pre-incubation mix (D) amount of undigested starch in the culture medium 24 hr after the exposure to the pre-incubation mix (E). Data are presented as mean±SE (n=28).

FIGS. 4.3(A)-(E): Effect of different silicas on cariogenic bacterial growth in the culture medium containing 1% starch. Silica 3, 5, and 7 were pre-incubated with human salivary amylase. The pre-incubation mix was added to S. mutans culture containing 1% starch. Several measurements were taken: bacterial growth for the first 8 hr (A-C), bacterial mass 24 hr after the addition of the pre-incubation mix (D), amount of undigested starch in the culture medium 24 hr after the addition of the pre-incubation mix (E). Data are presented as mean±SE (n=11).

FIG. 5: Effect of different silicas on cariogenic bacterial biofilm formation. Silica 3, 5, and 7 were pre-incubated with human salivary amylase. Amylase in the pre-incubation mix was added to S. mutans culture containing 1% starch. Several measurements were taken: bacterial growth 24 hr after the addition of the pre-incubation mix (A), amount of undigested starch in the culture medium 24 hr after the addition of the pre-incubation mix (B), and amount of biofilm formed 24 hr after the addition of the pre-incubation mix (C). Data are presented as mean±SE (n=22).

### EXAMPLES

The present invention will be further described by reference to the following examples, which are not intended to limit the scope of the invention.

### Example 1: Preparation/Characterization of Porous Silica Materials

### Materials

Silica 1, Silica 2 and Silica 3 were manufactured according to a process previously described (see Waara, E R et al., Adv. Healthcare Mater. 9(11), e2000057 (2020) and Baek, J et al., Nanomedicine (2021), in particular the experimental procedures described therein).

In brief, a meso-structure templating agent (P123, a triblock copolymer with average molecular weight=5800 g mol⁻¹, PEO₂₀PPO₇₀PEO₂₀) was dissolved in aqueous hydrochloric acid (HCl), with acid concentration equivalent to 1.6 M. Complete dissolution of P123 was followed by addition of tetraethyl orthosilicate (TEOS) under vigorous stirring at 40° C. The final molar ratio of P123: TEOS in the solution was 0.02:1.00 and the molar ratio of TEOS: HCl: H₂O was 1:6:235 (Silica 1), 1:7:250 (Silica 2) or 1:7:230 (Silica 3). The synthesis was kept static at 40° C. for 20 h and further hydrothermally treated for 20 h at 100° C. (Silica 1), 1.3 h at 85° C. (Silica 2) or 10 h at 100° C. (Silica 3).

Silica 4 was Kromasil 100-13-SIL purchased from Nouryon Pulp and Performance Chemicals AB.

Silica 5 (Sylodent) was Sylodent SM850C, a non-porous silica commonly used in toothpaste, obtained from W. R. Grace & Co.

Silica 6 was Sunsphere H-31 obtained from AGC Si-Tech.

Silica 7 was Sunsphere NP-30 obtained from AGC Si-Tech.

Silica 8 was LO-VEL 6200 obtained from PPG Industries, Inc.

### Nitrogen Sorption Analysis

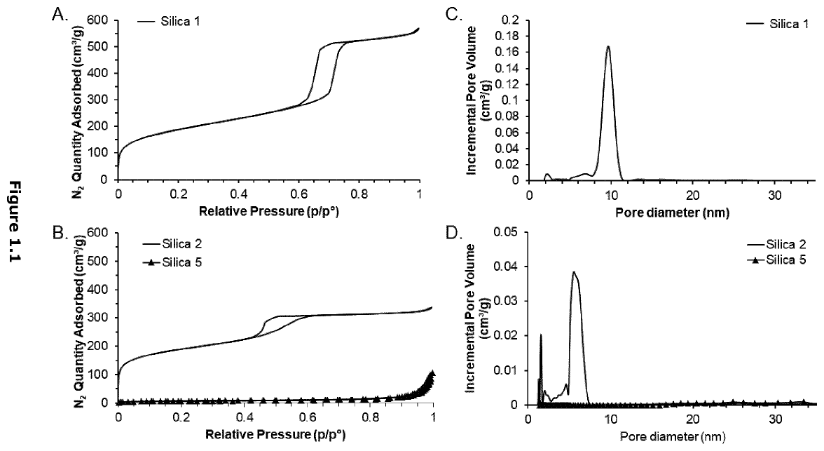

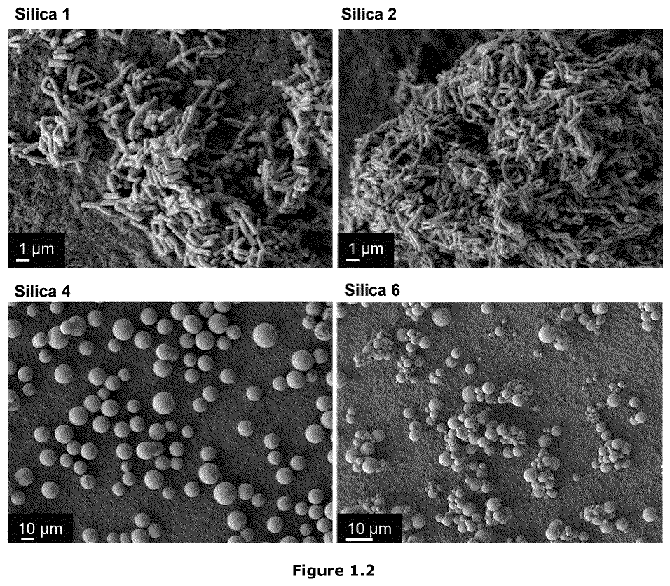

Brunauer-Emmett-Teller (BET) surface area was calculated from sorption isotherm at a relative pressure (p/p°) of <0.2 (plots in FIG. 1.1A and FIG. 1.1B). Total pore volume were recorded at a relative pressure (p/p°)=0.995. Average pore size and pore size distributions were derived from the adsorption curves using the Density Functional Theory (DFT) method or from the desorption curves using the Barrett-Joyner-Halenda (BJH) method. The pore size distribution data for the silica particles derived from the adsorption curves using DFT and a cylindrical pores-oxide surface model is presented in FIG. 1.1C and FIG. 1.1D. The measurements were performed at liquid nitrogen temperature (−196° C.) using a TriStar II volumetric adsorption analyser and data analysis was performed using the software MicroActive for TriStar II version 2.03 (Micromeritics Instrument Corp., GA, USA).

### Particle Size

Scanning electron microscopy using a JSM-7401F (JEOL Ltd., Tokyo, Japan) was used to characterize the particle size and morphology from SEM micrographs (FIG. 1.2). The mean particle size (length and width for rod-shaped particles Silica 1, Silica 2 and Silica 3 and diameter for spherical particles Silica 4, Silica 6 and Silica 7) were analyzed from ≥50 particles using ImageJ (Fiji; see Schindelin J, Arganda-Carreras I, Frise E et al., Fiji: an open-source platform for biological-image analysis., Nat. Methods, 9(7), 676-682 (2012)).

TABLE 1
Material characteristics
Various properties of the studied silica materials were measured using
above listed techniques with operational conditions. The features
identified are described in Table 1 below.

| | | | | Average | Average | | | | |
|---|---|---|---|---|---|---|---|---|---|
| | | | Average | pore size | pore size | | | % of | % of |
| | BET | Total | pore | in the | in the | Mean | % pores | mesopores | mesopores |
| | surface | pore | size - all | mesoporous | mesoporous | particle | in the | in the | in the |
| | area | volume | pores ¹ | range -DFT | range - BJH | size | mesoporous | 7-25 nm | 7-18 nm |
| | (m²/g) | (cm³/g) | (nm) | (nm)² | (nm)³ | (μm) | range ⁴ | range ⁵ | range ⁶ |
| Silica 1 | 687 | 0.87 | 5.1 | 8.8 | 6.3 | 0.9 | 91 | 75 | 74 |
| | | | | | | (length) | | | |
| | | | | | | 0.6 | | | |
| | | | | | | (width) | | | |
| Silica 2 | 695 | 0.52 | 3.0 | 5.2 | 3.9 | 1.4 | 66 | 2 | 2 |
| (negative | | | | | | (length) | | | |
| control) | | | | | | 0.2 | | | |
| | | | | | | (width) | | | |
| Silica 3 | 925 | 1.19 | 5.2 | 10.2 | 6.6 | 1.1 | 80 | 73 | 71 |
| | | | | | | (length) | | | |
| | | | | | | 0.3 | | | |
| | | | | | | (width) | | | |
| Silica 4 | 315 | 0.91 | 11.4 | 16.4 | 10.0 | 12.6 | 98 | 97 | 59 |
| | | | | | | (diameter) | | | |
| Silica 5 | 26 | | | | | not | | | |
| (Sylodent, | | | | | | measured | | | |
| negative | | | | | | | | | |
| control) | | | | | | | | | |
| Silica 6 | 746 | 0.77 | 4.1 | 6.8 | 4.6 | 4.1 | 90 | 42 | 42 |
| (negative | | | | | | (diameter) | | | |
| control) | | | | | | | | | |
| Silica 7 | 30 | | | | | 2.3 | | | |
| (negative | | | | | | (diameter) | | | |
| control) | | | | | | | | | |
| Silica 8 | 596 | 1.61 | 10.7 | 16.4 | 10.1 | not | 67 | 53 | 39 |
| (negative | | | | | | measured | | | |
| control) | | | | | | | | | |

¹ [4 × Total Pore Volume (cm³/g)/BET Surface Area (m²/g)] × 1000
²Calculated on adsorption curves, using DFT model and assuming a cylindrical pore geometry
³Calculated on desorption curves, using BJH model
⁴ Calculated from the DFT pore size distribution and total pore volume: [(Pore Volume at 50 nm − Pore Volume at 2 nm)/Total Pore Volume] × 100
⁵ Calculated from the DFT pore size distribution: [(Pore Volume at 25 nm − Pore Volume at 7 nm)/(Pore Volume at 50 nm − Pore Volume at 2 nm)] × 100
⁶ Calculated from the DFT pore size distribution: [(Pore Volume at 18 nm − Pore Volume at 7 nm)/(Pore Volume at 50 nm − Pore Volume at 2 nm)] × 100

Analysis of the properties of these silica materials is also provided in FIGS. 1.1(A)-(D) and FIG. 1.2 as described herein.

### Example 2: Carbohydrate Digestion Assay

### Preparation of Working Solutions and Standard Curve Samples

Prior to the assay, several working solutions were prepared. Firstly, 60-80 mg of mesoporous silica or control silica were weighed and dried overnight at 120° C. On the next day, silica was weighed again to obtain precise post-dried weight and 20 mg/mL silica suspension was prepared using double distilled water (ddH₂O). For Silica 3, sonication was necessary for homogeneous suspension. Briefly, 2 mm microtip (Vibra cell) was fit into the sonicator (Vibra cell) and the silica suspension was sonicated for 3 min at 40% amplitude without pulse. After the sonication, silica suspension was mixed several times by inversion and inspected visually. If silica clumps were still present, sonication was repeated once more. Generally, two rounds of sonication resulted in a dispersion with no or minimal clumps remaining. 1× PBS was prepared by dissolving 1 PBS tablet (Medicago, 09-2052-100) in 200 mL ddH₂O. Once dissolved, pH was adjusted to 5.4. Lyophilized human salivary amylase was rehydrated in ddH₂O to make 40 mg/mL stock solution. A working solution (8 mg/mL) was freshly prepared on the day of the experiment by diluting the necessary amount of stock solution with 1× PBS (pH 5.4). Starch stock solution (6 mg/ml) was prepared by mixing pure starch powder (Generation Ucan) in 1× PBS (pH 5.4). Starch does not readily dissolve in PBS, therefore the starch solution was microwaved several times (10-20 sec each time) until it started to boil and there was no more starch powder settling down at the bottom. When all dissolved, the starch solution remained opaque. When performing the digestion part of the assay, the starch stock solution was equilibrated to room temperature before starting the digestion. The starch standard curve samples (300 μL each) were prepared by serial dilution using 1× PBS (pH 5.4). The concentrations of the standard curve samples were: 1.5, 0.75, 0.375, 0.1875, 0.0938, 0.0465, and 0 mg/mL. Following serial dilution, 75 μL of 1N HCl (Sigma Aldrich, 1090571000) was added to each standard curve sample. Stock solutions of surfactants (cocamidopropyl betaine, polysorbate 80 and polyoxyethylene(4)lauryl ether) (1.0 wt %) were prepared using ddH₂O.

### Preparation of Test Formulations

Test formulations were prepared by mixing 40 μl of silica suspension in water (20 mg/mL) with 140 μl stock solutions or stock gels (concentrations according to Table 2).

TABLE 2
Formulations tested in Carbohydrate Digestion Assay

| | Concentration (wt %) | | | | | |
|---|---|---|---|---|---|---|
| | | | Mouthwash 2/ | | | |
| | Mouthwash 1 | | Mouthwash 3 | | Toothpaste 1 | |
| | (MW1) | | (MW2/MW3) | | (TP1) | |
| | Stock | Silica | Stock | Silica | Stock | Silica |
| Ingredient | solution | suspension | solution | suspension | gel | suspension |
| Water (ddH₂O) | 69.50 | 75.83 | 68.50 | 75.06 | 48.60 | 59.58 |
| Glycerol | 20.00 | 15.56 | 20.00 | 15.56 | 20.00 | 15.56 |
| Xylitol | 10.00 | 7.78 | 10.00 | 7.78 | 10.00 | 7.78 |
| Non-porous | — | — | — | — | 20.00 | 15.56 |
| silica - | | | | | | |
| Silica 5 | | | | | | |
| (Sylodent) | | | | | | |
| Surfactant* | — | — | 1.00 | 0.78 | — | — |
| Xantham gum | — | — | — | — | 1.00 | 0.78 |
| Sodium benzoate | 0.20 | 0.16 | 0.20 | 0.16 | 0.10 | 0.08 |
| Sodium fluoride | 0.30 | 0.23 | 0.30 | 0.23 | 0.30 | 0.23 |
| Active silica^{‡} - | — | 0.44 | — | 0.44 | — | 0.44 |
| as specified | | | | | | |

*Polysorbate 80 in MW2, PEG-40 castor oil in MW3.
^{‡}Active silica will refer to the silica material of the invention, as described in Example 1 and specified in the results.

### Adsorption of Salivary Amylase by Silica

Prior to starch digestion step, each sample was firstly incubated with salivary amylase for 15 min at 37° C. for enzyme adsorption/entrapment.

For mouthwash formulations, the incubation mix was prepared by mixing 20 μL of salivary amylase solution (8 mg/mL) and 180 μL of mouthwash formulation containing silica (prepared according to Table 2) in 1.5 mL microcentrifuge tube. This incubation mix was then incubated for 15 min at 37° C. with vertical rotation using a rotator (Harvard Apparatus, 74-2302).

For toothpaste formulation, the incubation mix was prepared by mixing 15 μL of salivary amylase solution (8 mg/mL) and 135 μL of toothpaste formulation containing silica (prepared according to Table 2) in 1.5 mL microcentrifuge tube. This incubation mix was then incubated in a water bath at 37° C. with horizontal shaking (200 rpm).

For testing of individual surfactant solutions, stock solutions of surfactants (cocamidopropyl betaine, polysorbate 80 and polyoxyethylene(4)lauryl ether) (1.0 wt %) were diluted to give the following concentrations using ddH₂O: 0.5, 0.25, 0.1, 0.05 wt % and silica was dispersed in each solution at a concentration of 4 mg/mL. The incubation mix was prepared by mixing 20 μL of salivary amylase solution (8 mg/ml) and 180 μL of diluted surfactant solution containing silica (4 mg/mL) in 1.5 mL microcentrifuge tube. This incubation mix was then incubated for 15 min at 37° C. with vertical rotation using a rotator (Harvard Apparatus, 74-2302).

For testing of Silica 8, 40 μl of silica suspension in water (20 mg/mL) was mixed with 140 μl of 1× PBS solution to have a silica dispersion of 4 mg/mL. The incubation mix was prepared by mixing 20 μL of salivary amylase solution (8 mg/mL) and 180 μL of silica suspension (4 mg/mL) in 1.5 mL microcentrifuge tube. This incubation mix was then incubated for 15 min at 37° C. with vertical rotation using a rotator (Harvard Apparatus, 74-2302).

Following the incubation, the diluted incubation mix was prepared by mixing 150 μL of the incubation mix with 1.45 ml of ddH₂O and 3.4 ml of 1× PBS (pH 5.4). For each carbohydrate digestion assay, a blank sample (No silica sample) was always included to monitor basal digestion level. For this sample, ddH₂O was added to the test formulation instead of silica suspension.

### Starch Digestion

The starch digestion was carried out for various durations: 0, 5 or 20 min. The reaction mix (800 μL) was prepared for each time point, which contained 1:1 ratio of starch (6 mg/mL) and diluted incubation mix. For each time point, 400 μL of starch stock solution (6 mg/mL) was aliquoted in 1.5 mL microcentrifuge tube. Then, 400 μL of diluted incubation mix was added to each tube. For the 0 min time point, 200 μL of 1N HCl was firstly added to starch solution, then 400 μL of diluted incubation mix was added. Each reaction mix was immediately mixed by inversion then incubated in a water bath at 37° C. with horizontal shaking (200 rpm) for each respective duration. When incubation was complete, 200 μL of 1N HCl was immediately added to terminate digestion.

### Colorimetric Determination of Digested Starch

The amount of digested starch was quantified at different time points using 5 mM iodine (Merck, 1.09099.1003). Each reaction mix was briefly vortexed before aliquoting into the 96-well plate (Corning, CLS3370). 75 μL of each reaction mix (silica samples as well as standard curve samples) was aliquoted in duplicate. Into each well, 75 μL of 5 mM iodine solution was aliquoted using a multi-channel pipette, then the absorbance was read at 570 nm.

### Calculation of the Amount of Digested Starch

The amount of the digested starch illustrates the efficacy of a given silica to reduce the digestion. The concentration of the undigested starch in each sample was extrapolated from the slope and the intercept of the starch standard curve. The percentage of undigested starch was then calculated by taking the 0 min time point as the 100% undigested starch. The percentage of digested starch was calculated by subtracting the percentage of undigested starch from 100. The percentage of digested starch at 5 min (every digestion assay except for testing of Silica 8) or 20 min (testing of Silica 8) time point was plotted as a bar graph. For every digestion assay, No silica sample was included which represented the basal digestion level.

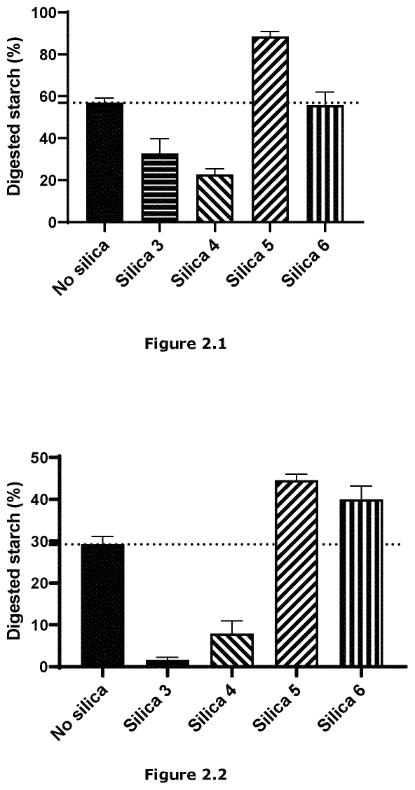

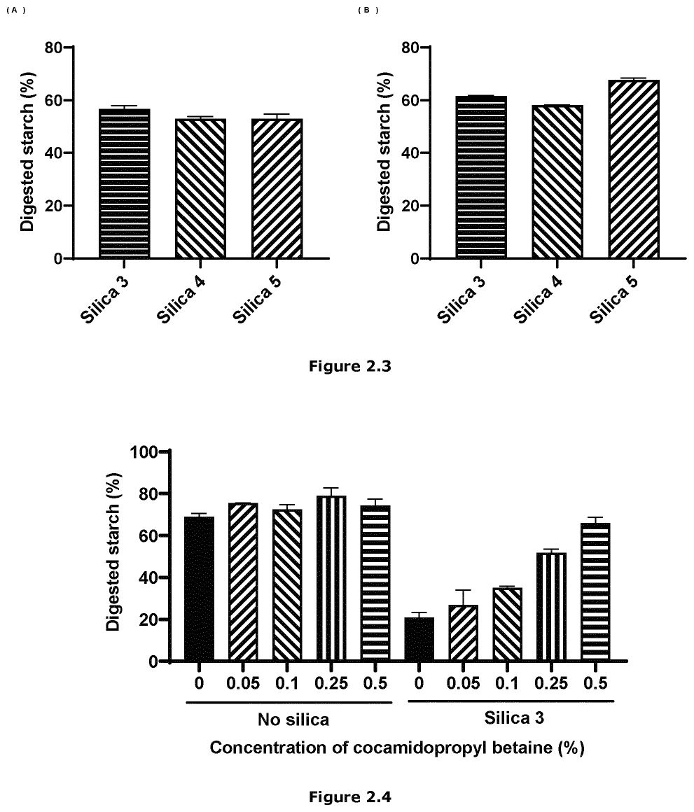

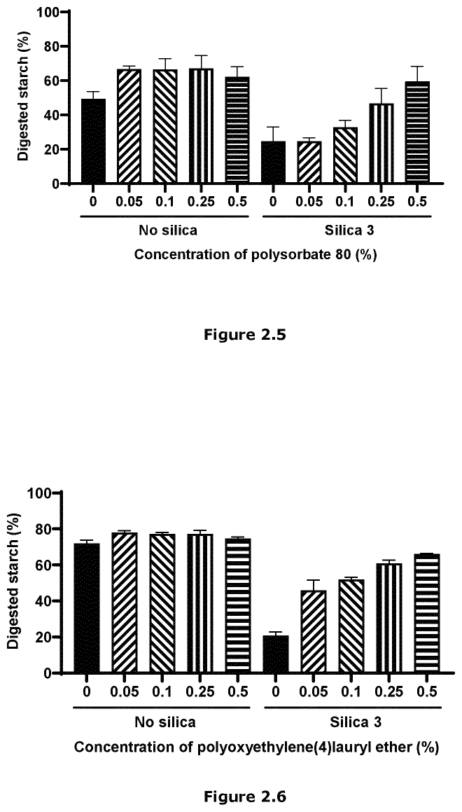

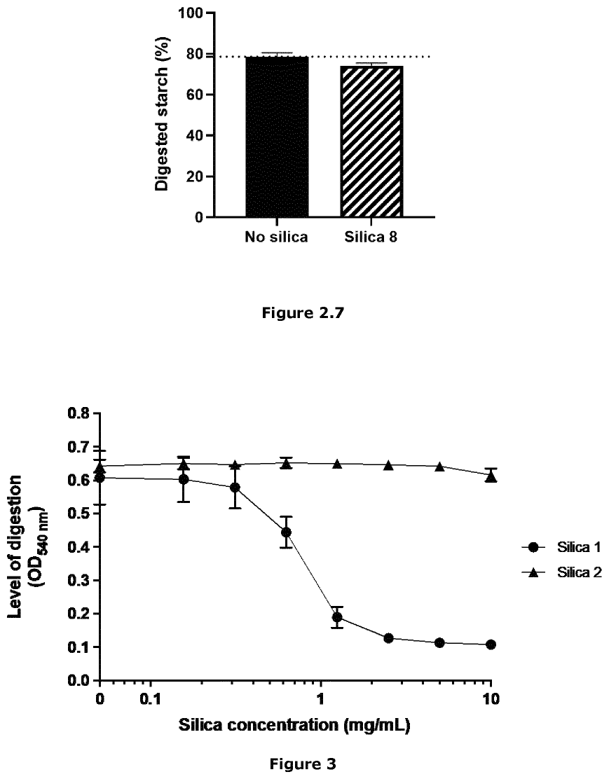

Results of these experiments are also presented in FIG. 2.1, FIG. 2.2, FIGS. 2.3(A)-(B), FIG. 2.4, FIG. 2.5, FIG. 2.6, and FIG. 2.7 as described herein.

### Example 3: Carbohydrate Digestion Assay Using Human Saliva

### Preparation of 2× PBS

2× PBS was prepared by dissolving 2 PBS tablets (Medicago, 09-2052-100) in 200 mL double distilled water (ddH₂O). Once dissolved, pH was adjusted to 5.4.

### Collection and Preparation of Human Saliva

Human saliva (2-3 mL) was collected in a test tube. Immediately after collection, saliva samples were centrifuged at 5000 rpm for 5 min to remove any particles or sediments. The supernatant (250 μL per sample) was collected and stored at −20° C. until the analysis.

On the day of analysis, saliva sample was thawed and 1:50 dilution was made using 2× PBS (pH 5.4). This was referred to as a saliva working solution.

### Preparation of Silica and its Working Solution

Approximately 60-80 mg of mesoporous silica was weighed and dried overnight at 120° C. On the next day, silica was weighed again to obtain precise post-dried weight and 20 mg/mL silica suspension was prepared using ddH₂O. Sonication of silica suspension was needed for homogeneous dispersion of silica. A microtip (Vibra cell, 630-0423) was fitted into the sonicator (Vibra cell, VCX 130) and silica suspension was sonicated for 3 min at 40% amplitude without pulse. Generally, two rounds of sonication resulted a homogenous solution with no or minimal clumps remaining.

### Preparation of Starch Solution and DNS Reagent

Starch solution (3 mg/mL) was prepared by mixing pure starch powder (Sigma Aldrich, 33615) in 1× PBS (pH 7.4). Starch does not readily dissolve in PBS, therefore the starch solution was microwaved several times (10-20 sec each time) until it started to boil and there was no more starch powder settling down at the bottom. When all dissolved, the starch solution remained opaque. When performing the digestion part of the assay, the starch solution was equilibrated to room temperature before starting the digestion. 3,5-dinitrosalicylic acid (DNS, Sigma Aldrich, D0550; 0.2 μM) was prepared by dissolving 1 g of DNS in 20 mL of 2 M NaOH. Into this solution, 30 g of sodium tartrate was added. The solution was topped up with ddH₂O to a final volume of 100 mL. The solution was stirred constantly using a magnetic stirrer and heated at 50° C. for 2 hr. The final solution was filtered and stored at 4° C.

### Adsorption of Salivary Amylase by Silica

Different silica concentrations (0.312-20 mg/mL, 60 μL each) was prepared by serial dilution in 96-well PCR plate (VWR, 732-2387) using ddH₂O. Sixty μL of saliva working solution was aliquoted into each well. The plate was sealed (Bio-rad, MSB1001) and incubated at 37° C. for 30 min with rotation using a rotator (Harvard Apparatus, 74-2302). When incubation was completed, the plate was centrifuged at 2000× g for 5 min at room temperature. The supernatant (30 μL) from each well was transferred to a new 96-well PCR plate.

### Colorimetric Determination of Reducing Sugars

Into each well containing the supernatant, 30 μL of starch solution (3 mg/mL) was added. The plate was sealed and incubated at 37° C. for 30 min with rotation using a rotator. Once incubation was finished, 60 μL of DNS solution (0.2 μM) was added into each well. The plate was sealed and incubated again at 95° C. for 7 min using a PCR machine. Following the final incubation, 100 μL of the solution was transferred to a 96-well plate (Corning, CLS3370) and the absorbance was read at 540 nm.

Results of these experiments are also presented in FIG. 3 as described herein.

### Example 4: Cariogenic Bacterial Growth Measurement

### Preparation of Working Solutions

1× PBS: 1× PBS was prepared by dissolving 1 PBS tablet (Medicago, 09-2052-100) in 200 mL double distilled water (ddH₂O). Once dissolved, pH was adjusted to 5.4. The solution was subsequently autoclaved (121° C., 20 min).

Brain Heart Infusion (BHI) broth: 37 g of BHI powder (BD Diagnostic Systems, 237500) was dissolved in 1 L of ddH₂O. To prepare BHI broth containing 1% starch, 10 g of starch powder (Generation Ucan) was added in the BHI broth solution. The broths were subsequently autoclaved (121° C., 20 min).

Starch stock solution (6 mg/mL): To prepare a starch stock solution, starch powder was sterilized by heating at 120° C. for 6 hr. The sterilized starch powder was added in autoclaved 1× PBS (pH 5.4) to make 6 mg/mL stock solution. Since starch does not readily dissolve in PBS, the solution was microwaved several times (10-20 sec each time) until it started to boil and there was no more starch powder settling down at the bottom. When all dissolved, the starch solution remained opaque.

Silica (20 mg/mL): 60-80 mg of mesoporous silica or control silica were weighed and dried overnight at 120° C. On the next day, silica was weighed again to obtain precise post-dried weight and 20 mg/ml silica suspension was prepared using autoclaved ddH₂O. For Silica 3, sonication was necessary for homogeneous suspension. Briefly, 2 mm microtip (Vibra cell) was fit into the sonicator (Vibra cell) and the silica suspension was sonicated for 3 min at 40% amplitude without pulse. After the sonication, silica suspension was mixed several times by inversion and inspected visually. If silica clumps were still present, sonication was repeated once more. Generally, two rounds of sonication resulted a dispersion with no or minimal clumps remaining.

Human salivary amylase: Lyophilized human salivary amylase (Sigma Aldrich, A1031) was rehydrated in ddH₂O to make 40 mg/mL stock solution. A working solution (8 mg/mL) was freshly prepared on the day of the experiment by diluting the necessary amount of stock solution in autoclaved 1× PBS (pH 5.4).

Preparation of Streptococcus mutans Culture

Streptococcus mutans (S. mutans) is one of the main cariogenic bacteria found in oral cavity that significantly contributes to dental caries. Lyophilized S. mutans (American Type Culture Collection (ATCC), 25175) was rehydrated in 5 mL of BHI broth. 0.5 mL of the suspension was aliquoted in four autoclaved test tubes and 4.5 mL of BHI broth was subsequently added in all test tubes. The inoculated tubes were incubated at 37° C. with constant agitation (shaker set at 120 rpm) for at least 30 hr. Following incubation, cultured samples were stored at −80° C. in 10 or 20% glycerol (Sigma Aldrich, G9012). S. mutans was cultured in BHI broth or in BHI broth containing 1% starch. S. mutans in 10% glycerol stock was used to inoculate 45 mL of the respective broth and cultured for 24-30 hr at 37° C. with constant agitation. The culture was kept at 4° C. until the day of the experiment, however not more than 48 hr. On the day of the experiment, 40 mL of the respective broth was added in the culture and incubated for 1 hr at 37° C. on a rotating platform. Following this incubation, the bacterial culture was diluted using the respective broth to adjust OD600 to approximately 0.1 (early logarithmic phase).

### Adsorption of Salivary Amylase by Silica

Prior to starch digestion step, each silica sample was firstly incubated with salivary amylase for enzyme adsorption. The incubation mix was prepared by mixing 140 μL of 1× PBS (pH 5.4), 40 μL of silica (4 mg/mL) and 20 μL of salivary amylase working solution (8 mg/mL) in 1.5 mL microcentrifuge tube. As a control, a blank sample (No silica sample) was always included, in which ddH₂O was added instead of silica. The incubation mix was incubated for 15 min at 37° C. with vertical rotation using a rotator (Harvard Apparatus, 74-2302). Following the incubation, 150 μL of the incubation mix was diluted in 1.48 mL ddH₂O and 3.4 mL 1× PBS (pH 5.4).

Starch Digestion and Feeding S. mutans With Starch Digestion Products

400 μL of diluted incubation mix was mixed with 400 μL of autoclaved starch stock solution (6 mg/mL) and incubated in the water bath at 37° C. with horizontal shaking (200 rpm) for 10 min. Subsequently, the samples were incubated at 95° C. for 45 min to deactivate salivary amylase. Following deactivation, the samples were centrifuged at 5000 rpm for 5 min at room temperature. The supernatant was then transferred to new 1.5 ml microcentrifuge tubes. Final concentrations in this reaction mix were: 60 μg/mL silica, 12 μg/mL salivary amylase, 3 mg/ml starch.

150 μL of the bacterial culture was aliquoted in a 96-well plate (Corning, CLS3370) and 50 μL of the reaction mix was added into each well. As a control, 100 μL of 1× PBS was added instead of the reaction mix. The culture was incubated at 37° C. for 24 hr with constant agitation. During the first 8 hr, bacterial growth was monitored by OD600 measurement every 30 min. After 24 hr, final OD600 was measured. The amount of undigested starch was also quantified after 24 hr by colorimetric measurement using iodine.

Growing S. mutans While Starch Digestion Occurs in Culture in Real-Time

In this set up, S. mutans was grown in BHI broth containing 1% starch. The digestion of starch in BHI broth occurred in real-time when the diluted salivary amylase-silica incubation mix was added to the bacterial culture. Adsorption of salivary amylase by silica was performed as described above. Following enzyme adsorption by silica, the incubation mix was centrifuged at 5000 rpm for 5 min at room temperature. Then 150 μL of the supernatant was diluted in 1.48 mL ddH₂O and 3.4 mL 1× PBS (pH 5.4) to prepare the diluted salivary amylase-silica incubation mix. In a 96-well plate, 150 μL of the bacterial culture (in BHI broth with 1% starch) was aliquoted in each well then 50 μL of the diluted salivary amylase-silica incubation mix was added. As a control, 50 μL of 1× PBS (pH 5.4) was added instead of the incubation mix. The culture was incubated at 37° C. with constant agitation up to 24 hr. During the first 8 hr, bacterial growth was monitored by OD600 measurement every 30 min, and final OD600 measurement was read after 24 hr. To quantify the amount of undigested starch in culture broth throughout incubation period, a plate was prepared for each representative incubation time point (0, 4, 8, and 24 hr). The amount of undigested starch was quantified by colorimetric measurement using iodine.

### Colorimetric Determination of Undigested Starch

The amount of undigested starch in culture broth was quantified by using iodine (Merck, 1.09099.1003). Briefly, 30 μL of culture broth was transferred to a new 96-well plate, then 45 μL of ddH₂O was aliquoted into each well. Then, 75 μL of 5 mM iodine was added to all wells and the absorbance was read at 570 nm.

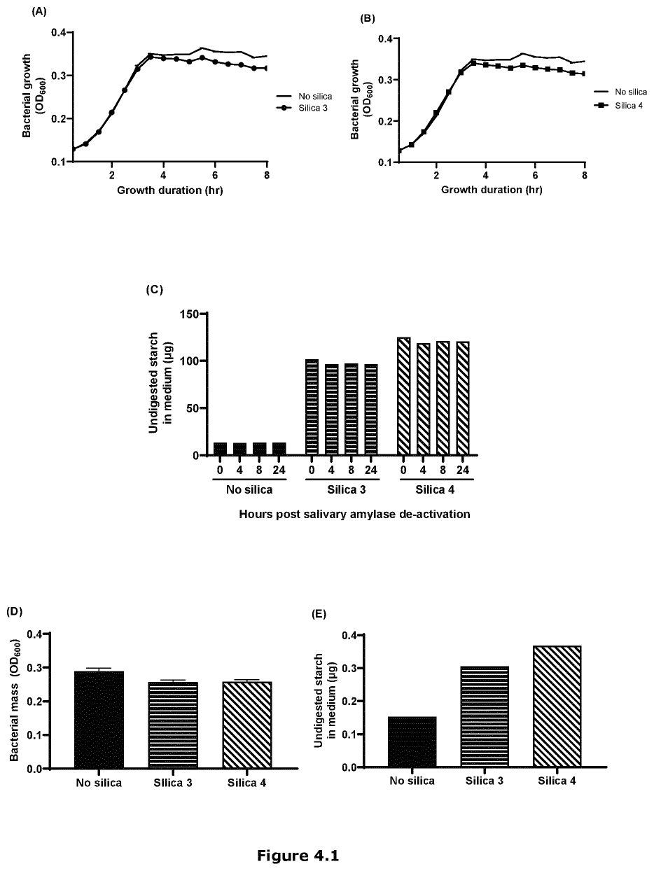

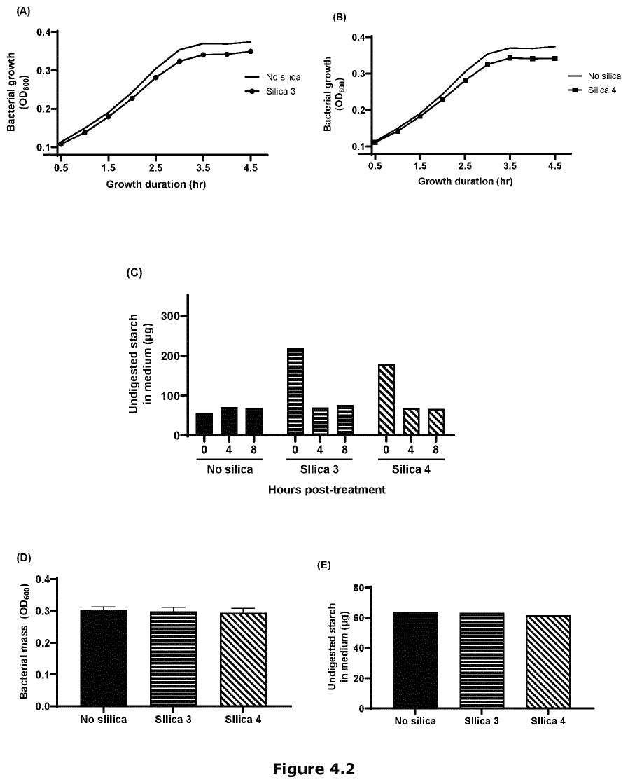

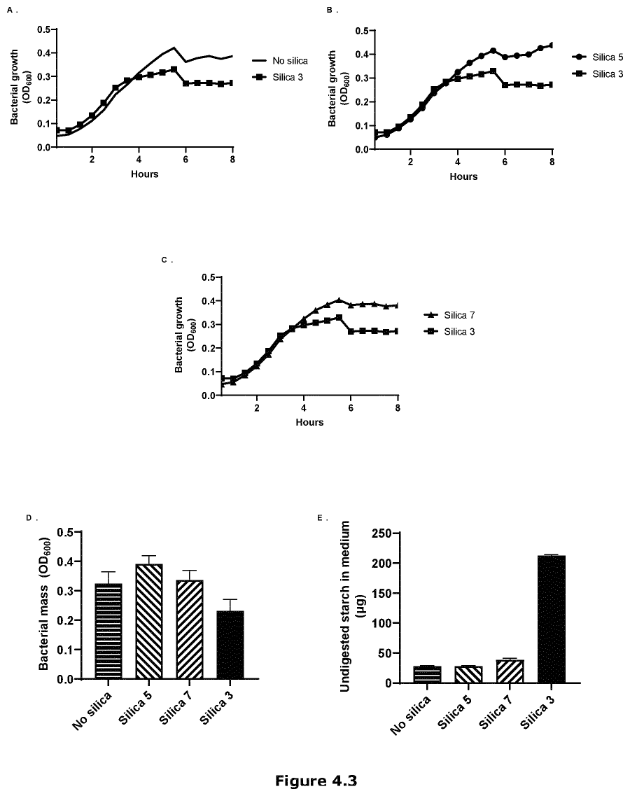

Results of these experiments are also presented in FIGS. 4.1(A)-(E), FIGS. 4.2(A)-(E), and FIGS. 4.3(A)-(E) as described herein.

### Example 5: Biofilm Formation Assay

### Preparation of Bacteria Cultures for Biofilm Formation

Streptococcus mutans bacterial culture was prepared and plated in a 96-well plate (Corning, CLS3370). 50 μL of the pre-incubation reaction mix was added in each well as described in the bacterial growth assessment. The culture was incubated at 37° C. for 24 hr without agitation.

### Colorimetric Determination of Biofilm Mass

After the 24-hr incubation, S. mutans culture medium was removed by inversion of the plate and the wells were washed with 150 μL of 1× PBS. The plate was inverted to discard the PBS and any remaining solution in each well was removed using a 200 μL pipette. 200 μl of methanol (Sigma Aldrich, 179957) was added in all wells to fix the biofilm and the plate was incubated statically at RT for 20 min. After removal of methanol by plate inversion, the plate was left open at RT for approximately 10 min to let any remaining methanol in each well to evaporate. After methanol had evaporated and the wells were dried, 200 μL of 0.002% crystal violet solution was added in each well to stain the biofilm. The 0.002% crystal violet solution was prepared by diluting 1% crystal violet stock solution (Sigma Aldrich, V5265) with ddH2O. Once the 0.002% crystal violet was added, the plate was covered with aluminium foil and incubated statically at RT for 40 min. After the incubation, the 0.002% crystal violet solution was discarded by plate inversion and any remaining solution was removed using a 200 μL pipette. Into each well, 200 μL of 98% ethanol (Sigma Aldrich, 1009831011) was added then the plate was covered in aluminium foil and was incubated at RT for 30 min on a shaking platform. The amount of biofilm formed was assessed by measuring the OD in the wells immediately after the de-staining step with ethanol at a wavelength of 590 nm.

### Other Measurements

After the 24-hr incubation, the growth of bacteria was assessed as described above (by measuring the OD at 600 nm). After the measurement, 30 μL of culture medium was transferred to a new 96-well plate and 45 μL of ddH₂O was aliquoted in each well. To quantify the remaining undigested starch levels in the media 75 μL of 5 mM iodine was added and colorimetric measurement was done as described above.

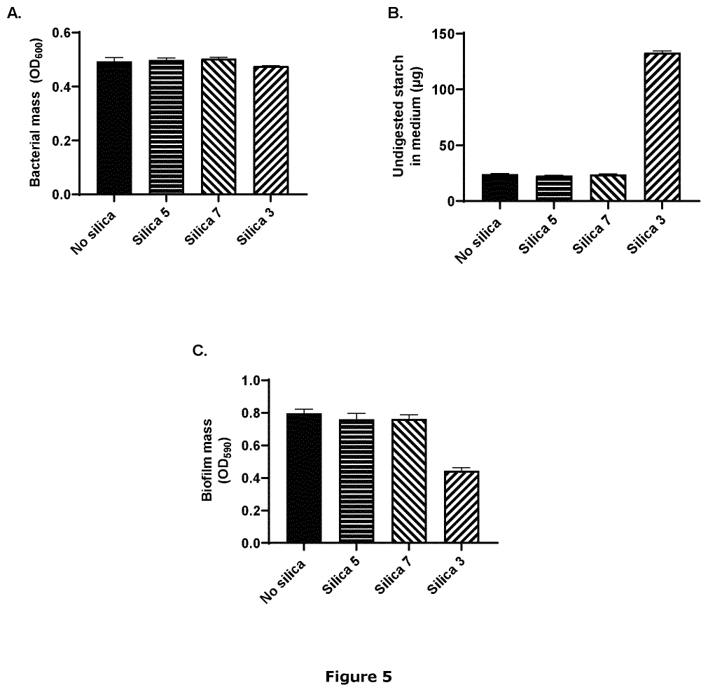

Results of these experiments are also presented in FIGS. 5(A)-(C) as described herein.

1. An oral care composition comprising porous silica particles having pores in the mesoporous range, wherein the average pore size of the pores in the mesoporous range is from about 7.0 to about 25.0 nm.

2. A composition according to claim 1, wherein the oral care composition is a dentifrice.

3. A composition as claimed in claim 1, wherein the composition is a toothpaste, a toothpowder or a mouthwash.

4. A composition as claimed in any one of claims 1 to 3, wherein the composition is substantially free of surfactant.

5. A composition as claimed in any one of claims 1 to 4, wherein the average pore size of the pores in the mesoporous range is from about 7.0 to about 22.0 nm.

6. A composition as claimed in any one of claims 1 to 5, wherein the average pore size of the pores in the mesoporous range is from about 7.0 to about 20.0 nm.

7. A composition as claimed in any one of claims 1 to 6, wherein the average pore size of the pores in the mesoporous range is from about 8.0 to about 13.0 nm.

8. A composition as claimed in any one of claims 1 to 7, wherein at least about 40% by volume of the pores are in the mesoporous range.

9. A composition as claimed in any one of claims 1 to 8, wherein the silica particles have a BET surface area of at least about 200 m₂/g.

10. A composition as claimed in any one of claims 1 to 9, wherein the silica particles have a mean particle size of from about 0.1 to about 20.0 μm.

11. A composition as claimed in any one of claims 1 to 10, wherein the silica particles have a mean particle size of from about 1.0 to about 5.0 μm.

12. A composition as claimed in any one of claims 1 to 11, wherein the silica particles have a total pore volume from about 0.7 to about 1.3 cm³/g.

13. The use of an oral care composition as defined in any one of claims 1 to 12 in:
the prevention of dental caries, the accumulation of dental plaque, gum disease, periodontitis, and/or tooth loss; or
the treatment or prevention of infections of the mouth.

14. An oral care composition as defined in any one of claims 1 to 12 for use in:
the prevention of dental caries, the accumulation of dental plaque, gum disease, periodontitis, and/or tooth loss; or
the treatment or prevention of infections of the mouth.

15. A method of:
the prevention of dental caries, the accumulation of dental plaque, gum disease, periodontitis, and/or tooth loss; or
the treatment or prevention of infections of the mouth,
in a subject in need thereof comprising the step of using an effective amount of an oral care composition as defined in any one of claims 1 to 12.

16. The use of a composition as defined in any one of claims 1 to 12 as an oral care product.

17. The use of a composition as defined in any one of claims 1 to 12 in a method of cleaning the oral cavity.
